(19) United States
(12) Patent Application Publication
(10) Pub. No.: US 2019/0290663 A1
(43) Pub. Date: Sep. 26, 2019

(54) One-stop Surgical Method of Coronary Intervention Therapy Combined with Endovascular Aortic Repair

(71) Applicant: YEE TAU TONG, Beijing (CN)

(72) Inventors: YEE TAU TONG, Beijing (CN); CHEUNG SHUEN, Beijing (CN); MAN YEUNG, Beijing (CN); SIU TUNG YIP, Beijing (CN); MING YIU LEE, Beijing (CN); KWAN FONG, Beijing (CN)

(21) Appl. No.: 16/361,572

(22) Filed: Mar. 22, 2019

(30) Foreign Application Priority Data
Mar. 22, 2018 (CN) 201810238906.5
Mar. 22, 2018 (CN) 201810238907.X

(51) Int. Cl. A61K 31/616; A61F 2/95; A61B 17/04; A61F 2/06; A61M 25/10; A61J 15/00; A61M 5/14; A61K 31/4365

(52) U.S. Cl. CPC A61K 31/616; A61F 2/95; A61B 17/04; A61F 2/06; A61B 2017/00292; A61J 15/00; A61M 5/14; A61K 31/4365; A61M 25/104

(57) ABSTRACT

The present disclosure provides a one-stop surgical method of coronary intervention therapy combined with endovascular aortic repair. The present disclosure specifically provides for the sequential implementation of endovascular aortic repair and coronary intervention therapy in a patient. The present disclosure belongs to the field of medical treatment, and the implementation of the present disclosure has the following advantages: (1) one-stop treatment of two kinds of diseases not only avoids the contradiction in anti-platelet treatment during separate surgeries, but also minimise the potential risk of the separate surgeries; (2) the patient only undergoes one anesthesia and one surgical process for the treatment of two diseases, which is easier to be accepted psychologically; and (3) hospitalization expenses and total hospitalization duration are reduced, and medical resource consumption is reduced.

### FIELD

The present disclosure belongs to the field of medical treatment, and specifically comprises the sequential implementation of endovascular aortic repair and coronary intervention therapy in a patient.

### BACKGROUND ART

Patients with systemic atherosclerosis are widespread. With the aggravation of population aging in China, the burden of multiple atherosclerotic diseases has become increasingly fierce, and the concept of “pan vascular disease” has been put forward and widely concerned. These patients require more comprehensive evaluation and treatment.

Therefore, screening for coronary artery diseases is recommended for middle-aged and elderly patients with non-emergency aortic aneurysm, aortic ulcer or dissection to avoid missed diagnosis and reduce the risk of perioperative myocardial infarction caused by coronary heart disease.

Similarly, for patients to be treated with coronary intervention therapy, aortic lesions, such as thoracic aortic perforating ulcer and aortic dissection, should be further evaluated if necessary. In this case, the aortic CTA examination must be completed, and adequate preoperative examination is helpful to reduce missed diagnosis and reduce the risk of aortic rupture and bleeding.

Traditional separate surgeries: traditionally, for patients with both coronary heart disease and aortic disease (aortic aneurysm/aortic ulcer or dissection, etc.) requiring intervention therapy, endovascular aortic repair (EVAR) is usually performed in vascular surgery department and coronary intervention therapy in the department of cardiology. The two surgeries are performed independently.

The advantage of this traditional one-by-one visit mode is that only one lesion is treated in one surgery, and the surgery and anesthesia time are short, however, the drawbacks include:

(1) There are contradictions in treatment: anti-platelet needs to be strengthened prior to PCI surgery, which will increase the risk of aortic dissection or ulcer rupture and bleeding and may even be life-threatening; the risk of ischemia and even myocardial infarction due to untreated coronary heart disease is also a concern during endovascular aortic repair.

(2) The patient is successively hospitalized in two wards and underwent two anesthesia, two surgeries and two perioperative treatments one after another, which may lead to prolonged hospitalization time, increased hospitalization expenses, increased risks and increased consumption of medical resources.

We have started to explore the field of pan vascular diseases, but no literature has reported the one-stop intracavitary intervention therapy for patients with aortic disease complicated with coronary heart disease.

### SUMMARY OF DISCLOSURE

The purpose of the present disclosure is to provide a one-stop surgical method of coronary intervention therapy combined with endovascular aortic repair, wherein the method comprises performing endovascular aortic repair and coronary intervention therapy successively on a patient.

For the above-mentioned surgical method, the endovascular aortic repair is performed first, and then the coronary intervention therapy is completed on the same table.

For the above-mentioned surgical method, a surgery is performed during a single anesthesia of the patient, and the anesthesia is a general anesthesia or a local anesthesia.

For the above-mentioned surgical method, the endovascular aortic repair comprises placing an aortic stent.

For the above-mentioned surgical method, only the femoral artery is used as the approach in the surgery.

For the above-mentioned surgical method, a femoral artery incision is completely sutured after completion of the endovascular aortic repair, and a sheath is placed by re-puncturing via a proximal end of the femoral artery incision under direct vision.

For the above-mentioned surgical method, the sheath is selected from one or more of the arterial sheaths with diameters of 6 F, 7 F, and 8 F.

For the above-mentioned surgical method, the coronary intervention therapy is a coronary stent implantation or a percutaneous coronary balloon angioplasty.

For the above-mentioned surgical method, aspirin or clopidogrel or a combination thereof is administered to the patient prior to the surgery.

For the above-mentioned surgical method, a dose of the aspirin is 100 mg once a day and a duration of administration is 1 to 30 days; or a dose of the clopidogrel is 75 mg once a day and a duration of administration is 1 to 30 days.

For the above-mentioned surgical method, a loading dose of the aspirin is 300 mg and an administration time is within 24 hours prior to the surgery; or a loading dose of the clopidogrel is 300 mg and an administration time is within 24 hours prior to the surgery.

For the above-mentioned surgical method, one or more of clopidogrel, ticagrelor, cilostazol, tirofiban, unfractonated heparin, or bivalirudin are administered to the patient immediately prior to the coronary intervention therapy or during the coronary intervention therapy.

For the above-mentioned surgical method, a dose of the clopidogrel is 300 mg; or a dose of the ticagrelor is 180 mg; or a dose of the cilostazol is 100 mg.

For the above-mentioned surgical method, the clopidogrel, the ticagrelor or the cilostazol is administered via enteral administration.

For the above-mentioned surgical method, the enteral administration refers to a bolus injection of a drug, via a pre-indwelling gastric tube prior to the surgery, into the patient's gastrointestinal tract after grinding the drug and stirring the same uniformly with water.

For the above-mentioned surgical method, the tirofiban is administered intravenously, a dose of administration is 0.1 to 0.15 micrograms per kilogram of body weight per minute, and a duration of administration is 1 to 24 hours; or a dose of the heparin is 75 to 100 units per kilogram of body weight; or the dose of the heparin is adjusted to 50 to 75 units per kilogram of body weight for a patient in which the tirofiban is used in combination; or an administration method of the bivalirudin comprises an intravenous injection of 0.75 milligrams per kilogram of body weight and a continuous intravenous infusion of 1.75 milligrams per kilogram of body weight per hour.

For the above-mentioned surgical method, a monitoring of whole blood activated clotting time (ACT) is required during an administration of either the heparin or the bivalirudin, and the ACT is maintained between 250 and 350 seconds during the coronary intervention therapy.

For the above-mentioned surgical method, after completion of the coronary intervention therapy, a femoral artery puncture site is sutured under direct vision, and a muscular layer and a cortex are sutured after sufficient hemostasis.

For the above-mentioned surgical method, the approach of the femoral artery is surgically incised, and the femoral artery is punctured and sutured under direct vision.

For the above-mentioned surgical method, it is suggested that a postoperative hydration is performed on a patient with a total usage amount of an intraoperative contrast agent greater than 2 ml per kilogram of body weight.

For the above-mentioned surgical method, the hydration is performed by an intravenous infusion of 0.9% normal saline at a rate of 0.5 to 1 ml per kilogram of body weight per minute, and a hydration duration is 12 to 48 hours.

For the above-mentioned surgical method, if there is no postoperative bleeding complication, a dual anti-platelet treatment is maintained the next day after the surgery.

For the above-mentioned surgical method, wherein the dose of the aspirin is 75 to 100 mg/day and maintained for a long period; or the dose of the clopidogrel is 75 mg/day and maintained for 6 to 12 months; or the dose of the ticagrelor is 90 mg/time, twice a day, and maintained for 6 to 12 months; or the dose of the cilostazol is 100 mg/time, twice a day, and maintained for 6 to 12 months.

Nowadays, as medical disciplines become more and more specialized, we gradually realize that a patient should be considered as a whole. Our understanding of the disease should be based on the patient's perspective. If the same patient is affected by complicated multiple diseases, we should make multi-disciplinary cooperation, carry out comprehensive evaluation, and strive to provide individualized and one-stop medical services for the patient, which is also the development trend of modern healthcare.

For patients with both the aortic disease and the coronary artery disease, we have established a joint team of experts from departments of vascular surgery and cardiology, and have explored multiple cases of one-stop treatment by the one-stop surgical method of the coronary intervention therapy combined with the endovascular aortic repair. The advantages of this one-stop treatment are:

(1) one-stop treatment of two kinds of diseases not only avoids the contradiction in anti-platelet treatment during separate surgeries, but also avoids the potential risk brought by the other disease during the separate surgeries;

(2) the patient only undergoes one anesthesia and one surgical process for the treatment of two diseases, which is easier to be accepted psychologically; and

(3) hospitalization expenses are reduced, ICU duration and total hospitalization duration are shortened, and medical resource consumption is reduced.

However, the difficulties of this one-stop treatment are:

(1) it requires experts from two department to form a composite technical team, which requires high technical level and coordination ability of related disciplines of the hospital;

(2) when the two diseases are treated at the same stage, the total amount of the contrast agent used in a short time increases, which may increase the adverse reactions related to the contrast agent; and

(3) the perioperative period of the coronary intervention therapy requires dual anti-platelet therapy to reduce the risk of thrombosis; the approach of endovascular aortic repair (e.g., femoral artery surgery incision) is more prone to postoperative bleeding, subcutaneous ecchymosis, local hematoma, pseudoaneurysm and other bleeding risks.

Therefore, the connection between the endovascular aortic repair and the coronary intervention therapy, and perioperative antithrombotic therapeutic regimen are critical. In view of this problem, we developed a set of rational and feasible antithrombotic therapeutic regimen for the one-stop treatment by the one-stop surgical method of the coronary intervention therapy combined with the endovascular aortic repair. Moreover, taking perioperative massive hemorrhage as the primary observation endpoint, we also compared the safety of one-stop treatment by the one-stop surgical method of the coronary intervention therapy combined with the endovascular aortic repair with that of separate treatments.

Clinical trials for this project are already under way, and currently not less than 10 cases of one-stop treatment for coronary intervention therapy combined with endovascular aortic repair have been completed. The specific implementation method is shown in FIG. 2.

The implementation of the present disclosure has the following advantages: (1) one-stop treatment of two kinds of diseases not only avoids the contradiction in anti-platelet treatment during separate surgeries, but also avoids the potential risk of the separate surgeries; (2) the patient only experiences one anesthesia and one surgical process for the treatment of two diseases, which is easier to be accepted psychologically; (3) hospitalization expenses are reduced and total hospitalization duration is shortened, and medical resource consumption is reduced; and (4) it has a better long-term therapeutic effect on “pan vascular diseases”.

### BRIEF DESCRIPTION OF THE DRAWINGS

FIG. 1 shows the schematic diagram of the aorta and coronary arteries.

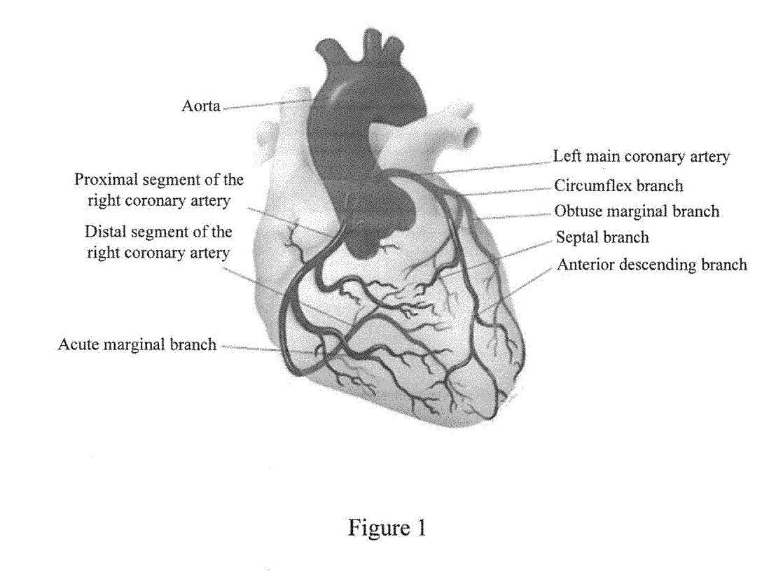

FIG. 2 shows the flow chart of the clinical trial.

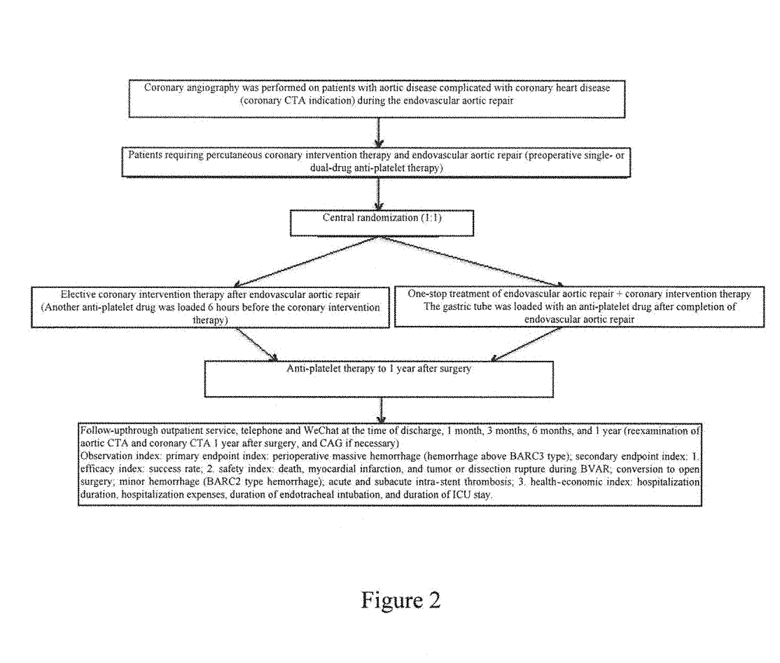

### DETAILED DESCRIPTION OF EMBODIMENTS

Experimental examples of a one-stop surgical method of coronary intervention therapy combined with endovascular aortic repair are provided below, and are intended to illustrate the present disclosure rather than limit it.

When used in the claims and/or the description, the word “a” or “an” or “the” may refer to “one”, but may also refer to “one or more”, “at least one” and “one or more than one”.

As used in the claims and the description, the words “comprising”, “having”, “including” or “containing” means inclusive or open-ended, and does not exclude additional, unrecited elements or method steps. Meanwhile, “comprising”, “having”, “including” or “containing” may also mean closed-ended, and excludes additional, unrecited elements or method steps.

### EXPERIMENTAL EXAMPLE 1

A 77-year-old male patient who was diagnosed with coronary heart disease complicated with aortic ulcer. Preoperatively, aspirin was administered orally at 100 mg once a day for 5 consecutive days. Endovascular aortic repair was performed under general anesthesia through the right femoral artery approach, in which a stent was implanted in the descending aorta. Subsequently, coronary angiography was performed on the same table through the same approach, indicating severe stenosis of the obtuse marginal branch. 300mg of clopidogrel was loaded via the gastric tube, followed by coronary intervention therapy in which a stent was implanted in the obtuse marginal branch. The surgery was successful, the postoperative recovery was good, no massive bleeding events occurred after operation, and no massive bleeding, acute myocardial infarction or other events occurred during the 1-year follow-up.

### EXPERIMENTAL EXAMPLE 2

A 72-year-old female patient who was diagnosed with coronary heart disease complicated with intramural aortic hematoma. Preoperatively, aspirin was administered orally at 100 mg once a day for 5 consecutive days. Endovascular aortic repair was performed under general anesthesia through the right femoral artery approach, in which a stent was implanted in the descending aorta. Subsequently, coronary angiography was performed through the same approach, indicating stenosis of the anterior descending branch of 80% Immediately, 300 mg of clopidogrel was loaded via the gastric tube, followed by coronary intervention therapy in which a stent was implanted in the anterior descending branch. The process went smoothly. After operation, aspirin and clopidogrel were taken regularly for anti-platelet treatment, no massive bleeding, acute myocardial infarction or other events occurred after operation, and no massive bleeding and acute myocardial infarction or other events occurred during the 1-year follow-up.

### EXPERIMENTAL EXAMPLE 3

A 62-year-old male patient who was diagnosed with coronary heart disease complicated with aortic dissection. Preoperatively, aspirin was administered orally at 100 mg once a day for 3 consecutive days. Endovascular aortic repair was performed under general anesthesia through the right femoral artery approach. Subsequently, coronary angiography was performed through the right radial artery approach, indicating stenosis of the anterior descending branch of 90%, and immediately 300 mg of clopidogrel was loaded via the gastric tube, followed by coronary intervention therapy in which a stent was implanted in the anterior descending branch. The process went smoothly. After operation, aspirin and clopidogrel were taken regularly for anti-platelet treatment, and no massive bleeding, acute myocardial infarction or other events occurred.

1. A one-stop surgical method of coronary intervention therapy combined with endovascular aortic repair, wherein the endovascular aortic repair and the coronary intervention therapy are performed successively on a patient.

2. The surgical method according to claim 1, wherein the endovascular aortic repair is completed first, and then the coronary intervention therapy is completed on the same table.

3. The surgical method according to claim 1, wherein a surgery is performed during a single anesthesia of the patient, and the anesthesia is a general anesthesia or a local anesthesia.

4. The surgical method according to claim 1, wherein the endovascular aortic repair requires placing an aortic stent.

5. The surgical method according to claim 1, wherein the coronary intervention therapy gains access including one or more of the following: a femoral artery, a radial artery, a brachial artery, or an ulnar artery.

6. The surgical method according to claim 5, wherein a femoral artery incision is completely sutured after completion of the endovascular aortic repair, and a sheath is placed by re-puncturing via a proximal end of the femoral artery incision under direct vision.

7. The surgical method according to claim 6, wherein the sheath is selected from one or more of arterial sheaths with diameters of 6 F, 7 F, and 8 F.

8. The surgical method according to claim 1, wherein the coronary intervention therapy is a coronary stent implantation or a percutaneous coronary balloon angioplasty.

9. The surgical method according to claim 1, wherein aspirin or clopidogrel or a combination thereof is administered to the patient prior to the surgery.

10. The surgical method according to claim 9, wherein a dose of the aspirin is 100 mg once a day and a duration of administration is 1 to 30 days; or a dose of the clopidogrel is 75 mg once a day and a duration of administration is 1 to 30 days.

11. The surgical method according to claim 10, wherein a loading dose of the aspirin is 300 mg and an administration time is within 24 hours prior to the surgery; or a loading dose of the clopidogrel is 300 mg and an administration time is within 24 hours prior to the surgery.

12. The surgical method according to claim 1, wherein one or more of clopidogrel, ticagrelor, cilostazol, tirofiban, unfractionated heparin, or bivalirudin are administered to the patient immediately prior to or during the coronary intervention therapy.

13. The surgical method according to claim 12, wherein a dose of the clopidogrel is 300 mg; or a dose of the ticagrelor is 180 mg; or a dose of the cilostazol is 100 mg.

14. The surgical method according to claim 13, wherein the clopidogrel, the ticagrelor or the cilostazol is administered via enteral administration.

15. The surgical method according to claim 14, wherein the enteral administration refers to a bolus injection of a drug, via a pre-indwelling gastric tube prior to the surgery, into the patient's gastrointestinal tract after grinding the drug and stirring the same uniformly with water.

16. The surgical method according to claim 12, wherein the tirofiban is administered intravenously, a dose of administration is 0.1 to 0.15 micrograms per kilogram of body weight per minute, and a duration of administration is 1 to 24 hours; or a dose of the heparin is 75 to 100 units per kilogram of body weight; or the dose of the heparin is adjusted to 50 to 75 units per kilogram of body weight for a patient in which the tirofiban is used in combination; or an administration method of the bivalirudin comprises an intravenous injection of 0.75 milligrams per kilogram of body weight and a continuous intravenous infusion of 1.75 milligrams per kilogram of body weight per hour.

17. The surgical method according to claim 12, wherein a monitoring of whole blood activated clotting time (ACT) is required during an administration of either the heparin or the bivalirudin, and the ACT is maintained between 250 and 350 seconds during the coronary intervention therapy.

18. The surgical method according to claim 1, wherein after completion of the coronary intervention therapy, a femoral artery puncture site is sutured under direct vision, and a muscular layer and a cortex are sutured after sufficient hemostasis.

19. The surgical method according to claim 1, wherein the approach of the femoral artery is surgically incised, and the femoral artery is punctured and sutured under direct vision.

20. The surgical method according to claim 1, wherein a postoperative hydration is performed on a patient with a total usage amount of an intraoperative contrast agent greater than 2 ml per kilogram of body weight.

21. The surgical method according to claim 20, wherein the hydration is performed by an intravenous infusion of 0.9% normal saline at a rate of 0.5 to 1 ml per kilogram of body weight per minute, and a hydration duration is 12 to 48 hours.

22. The surgical method according to claim 1, wherein if there is no postoperative bleeding complication, a dual anti-platelet treatment is maintained the next day after the surgery.

23. The surgical method according to claim 1, wherein the dose of the aspirin is 75 to 100 mg/day and maintained for a long period; or the dose of the clopidogrel is 75 mg/day and maintained for 6 to 12 months; or the dose of the ticagrelor is 90 mg/time, twice a day, and maintained for 6 to 12 months; or the dose of the cilostazol is 100 mg/time, twice a day, and maintained for 6 to 12 months.
